SEQUENCE LISTING (30 sequences)

SEQ ID NO: 1 - DNA, 24 bp, artificial sequence
  forward primer for Id2
tctgagcttatgtcgaatgatagc

SEQ ID NO: 2 - DNA, 24 bp, artificial sequence
  reverse primer for Id2
cacagcattcagtaggctcgtgtc

SEQ ID NO: 3 - DNA, 24 bp, artificial sequence
  forward primer for Cdx2
cccagcggccagcggcgaaacctg

SEQ ID NO: 4 - DNA, 25 bp, artificial sequence
  reverse primer for Cdx2
ttctcgcagcgtccatactcctcat

SEQ ID NO: 5 - DNA, 20 bp, artificial sequence
  forward primer for ERRBeta
tcaagtgcgagtacatgctt

SEQ ID NO: 6 - DNA, 20 bp, artificial sequence
  reverse primer for ERRBeta
gaaatctgtaagctcaggta

SEQ ID NO: 7 - DNA, 20 bp, artificial sequence
  forward primer for Hand1
gcgcctggctaccagttaca

SEQ ID NO: 8 - DNA, 20 bp, artificial sequence
  synthetic construct
agcaacgccttccctctagg

SEQ ID NO: 9 - DNA, 21 bp, artificial sequence
  forward primer for Mash2
gaaggtgcaaacgtccacttc

SEQ ID NO: 10 - DNA, 21 bp, artificial sequence
  reverse primer for Mash2
ccttactcagcttcttgttgg

SEQ ID NO: 11 - DNA, 21 bp, artificial sequence
  forward primer for PL-1
tgactttgactctttcgggct

SEQ ID NO: 12 - DNA, 21 bp, artificial sequence
  reverse primer for PL-1
gctctgaatacaccgagagcg

SEQ ID NO: 13 - DNA, 17 bp, artificial sequence
  forward primer for Stra13
tttccagachtgtgccc

SEQ ID NO: 14 - DNA, 19 bp, artificial sequence
  reverse primer for Stra13
taccagcayttctccagca

SEQ ID NO: 15 - DNA, 21 bp, artificial sequence
  forward primer for 4311/TPBP
caggtacttgagacatgactc

SEQ ID NO: 16 - DNA, 22 bp, artificial sequence
  reverse primer for 4311/TPBP
ggcagagatttcttagacaatg

SEQ ID NO: 17 - DNA, 22 bp, artificial sequence
  forward primer for Tfeb
gcgcatgcagcagcaggctgtc

SEQ ID NO: 18 - DNA, 22 bp, artificial sequence
  reverse primer for Tfeb
ctggggatgctgctggggcagg

SEQ ID NO: 19 - DNA, 21 bp, artificial sequence
  forward primer for Tec
ataagaaagaccctgcctccc

SEQ ID NO: 20 - DNA, 20 bp, artificial sequence
  reverse primer for Tec
aagcctcaccactccaaaca

SEQ ID NO: 21 - DNA, 20 bp, artificial sequence
  forward primer for Gcm-1
agaccaagctggaagcagag

SEQ ID NO: 22 - DNA, 19 bp, artificial sequence
  reverse primer for Gcm-1
gcatgttgctgtgagtagg

SEQ ID NO: 23 - DNA, 18 bp, artificial sequence
  forward primer for Dix-3
atctcaatgggctcgcag

SEQ ID NO: 24 - DNA, 20 bp, artificial sequence
  reverse primer for Dix-3
atggagtcactgttgttggg

SEQ ID NO: 25 - DNA, 17 bp, artificial sequence
  forward primer for Esx-1
gcaaccccaacaggagc

SEQ ID NO: 26 - DNA, 18 bp, artificial sequence
  reverse primer for Esx-1
ggactcatggcgactgga

SEQ ID NO: 27 - DNA, 20 bp, artificial sequence
  forward primer for Beta Actin
atcgtgggccgccctaggca

SEQ ID NO: 28 - DNA, 20 bp, artificial sequence
  reverse primer for Beta Actin
tggccttagggttcagaggg

SEQ ID NO: 29 - DNA, 18 bp, artificial sequence
  forward primer for GAPDH
ggagtcaacggatttggt

SEQ ID NO: 30 - DNA, 20 bp, artificial sequence
  reverse primer for GAPDH
gtgatgggatttccattgat

(19) United States
(12) Patent Application Publication
(10) Pub. No.: US 2007/0259353 A1
(43) Pub. Date: Nov. 8, 2007

(54) RT-PCR profiling for the analysis of placental trophoblast cell lineages

(76) Inventors: Thomas Lee Brown, Beavercreek, OH (US); Amy Denise Gultice, Spring Valley, OH (US); Kaisa Leena Selesniemi, Boston, MA (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 11/429,920

(22) Filed: May 8, 2006

(51) Int. Cl. C12Q 1/68; C12P 19/34

(52) U.S. Cl. 435/6; 435/91.2

(57) ABSTRACT

A method of comprehensively identifying a specific placental cell lineage of a placental cell sample, applicable to all placental species, is provided. The method employs RT-PCR technology and comprises detecting expression markers unique for each of four trophoblast cell lineages using specifically designed inventive PCR primer pair panels. Diagnostic and prognostic methods, as well as kits and panels related thereto, are also provided.

### FIELD OF THE INVENTION

The present invention relates to the use of gene-specific oligonucleotide PCR primers that are uniquely expressed and can be used to identify particular cell lineages (types) of the placenta during development. Different placental cell lineages (trophoblasts) have very different cell functions. The present invention permits the analysis of trophoblast stem cells and monitoring of their differentiation and development into cells that, for example, may become invasive and serve to provide nutrients to the baby. The invention employs an analysis based on conserved gene expression and will therefore be useful in the analysis of placental development in many species.

### BACKGROUND

Reverse transcription-polymerase chain reaction (RT-PCR)-based cell analysis and gene-profiling is well-known in the art. It is commonly considered the most sensitive technique for mRNA detection and quantization currently available. Compared to two other commonly used techniques for quantifying mRNA levels, Northern blot analysis and RNase protection assay, RT-PCR can be used to quantify MRNA levels from much smaller samples. In fact, this technique is sensitive enough to enable quantization of RNA from a single cell. Reverse transcription, also known as “Real-time”-PCR has become the preferred method for validating results obtained from array analyses and other techniques that evaluate gene expression changes on a global scale.

Some application of RT-PCR to the analysis of cell differentiation is also known. For example, Stanton et al disclose a descriptive microarray (gene chip) of genes up regulated or down regulated during human Embryonic Stem (ES) Cell differentiation. However this work is directed to ES and not placental trophoblast and cell lineage determination is not addressed.

Rossant et al discloses methods for isolation, culture and differentiation of a particular cell line, and discloses limited cell lineage analysis of the placental cells. Rossant also discloses a method of isolating placental trophoblast stem cells and further discloses methods utilizing RT-PCR to show that cells are trophoblast stem cells and can differentiate into giant cells by lineage. However there is no analysis of labyrinthine lineage and no method or suggestion for providing a method for comprehensive analysis via a combination of positive results and elimination of alternatives. The labyrinthine placenta is the main placental zone, where nutrient exchange largely occurs.

There is currently a need in the art for methods of comprehensively analyzing placental cell lineage and identifying specific placental cell lineages in a rapid and cost-effective manner. There is further a need in the medical arts for diagnostic kits which provide rapid, routine and accurate analysis of placental cell lineages in order to monitor and improve the outcomes of pregnancies. Further, there is a need in the animal husbandry arts for methods of cost-effectively monitoring the status, health or progression of pregnancies to maximize the quality and quantity of offspring.

### SUMMARY OF THE INVENTION

Accordingly, the present invention utilizes RT-PCR technology in novel methods which enable the rapid determination of a particular cell lineage. A panel of placental lineage markers is provided to identify a specific lineage as positive and to eliminate doubt of alternate lineage by negative expression. The determination all four placental trophoblast lineages is determinable at one time.

One embodiment of the present invention is directed to a method of comprehensively identifying a specific placental cell lineage of a placental cell sample. The method comprises: (1) isolating RNA of the cell sample to yield a total RNA isolate and subjecting the total RNA isolate to reverse transcription PCR (RT-PCR) to yield a placental cell genetic sample; (2) dividing the genetic sample into at least four portions, including a first, a second, a third and a fourth portion; (3) testing the first portion for presence of a trophoblast stem cell lineage marker; (4) testing the second portion for presence of a trophoblast giant cell lineage marker; (5) testing the third portion for presence of a syncytiotrophoblast/labyrinthine cell lineage marker; (6) testing the fourth portion for DNA contamination; (7) conducting an analysis of the results wherein the specific placental cell lineage is determined. PCR primers which may be suitably employed in the present invention are those which detect the unique markers.

Placental cell samples may derive from a variety of species, including human, and the markers contemplated as suitable for inclusion in the present invention should be selected to enable comprehensive determination of cell lineage for a given species. Positive identification combined with positive elimination of alternatives, in the same test, provides a cost-effective and time-efficient method for determining placental cell lineage of a sample.

Another embodiment of the present invention is directed to a panel of PCR primer pairs suitable for detecting at least one placental cell genetic lineage marker from each of the following: trophoblast stem cell lineage markers, trophoblast giant cell markers, and syncytiotrophoblast/labyrinthine cell lineage markers. The panel may also include at least one PCR primer pair suitable for detecting a spongiotrophoblast cell lineage marker. Any PCR primer pair suitable for detecting a unique marker may be employed.

A further embodiment is directed to a kit which may be used to monitor pregnancy over the course of a pregnancy, or, for example, to detect placental abnormalities early in pregnancy in order to provide opportunity for effective early intervention. The kit comprises a panel according to the present invention, and may also comprise genetic amplification technology and other components related to expression and detection of the genetic marker. The suitability of such components would be readily ascertained by a person of ordinary skill in the art.

One component of the kit may comprise any means, known or unknown, for amplifying RNA, DNA or attaching protein to a matrix or plastic for use as a substrate, including but not limited to RT-PCR, Northern Blot, Miningene array, Western Blot analysis, enzyme-linked immunoassays (ELISA), and dipsticks. The kits are directly capable of determining placental lineage in a placental cell sample. The kits may also be useful for diagnosis of placental disorders, including but not limited to pre-eclampsia, intrauterine growth retardation and trophoblast neoplasms. In some embodiments the inventive methods are adapted to provide monitoring the status and progression of pregnancy in both humans and other animal species.

### DETAILED DESCRIPTION OF THE INVENTION

Cells of the trophoblast lineage make up the epithelial compartment of the placenta, and their rapid development is essential for the establishment and maintenance of pregnancy. A diverse array of specialized trophoblast subtypes form throughout gestation and are responsible for mediating implantation, as well as promotion of blood to the implantation site, changes in maternal physiology, and nutrient and gas exchange between the fetal and maternal blood supplies. Trophoblasts originate as stem cells, differentiate into genetically identifiable lineages, and establish the maternal-fetal interface. Distinct trophoblast lineages make the placenta an attractive model to understand the control of stem cell growth and differentiation.

The inability of trophoblast cells to undergo proper differentiation is associated with intrauterine growth retardation and preeclampsia. Trophoblasts, therefore, provide a model to investigate fundamental mechanisms of stem cell differentiation. However, the availability of trophoblast stem cell lines is limited.

Previously, several systems have been used to analyze the major placental functions. Purified human cytotrophoblast cells represent a trophoblast stem cell population and become invasive on Matrigel, representing the extravillous trophoblast lineage. When purified human cytotrophoblast cells are grown on tissue culture plates, they spontaneously differentiate and fuse representing the syncytiotrophoblast lineage. Placental explants have been used to examine trophoblast outgrowth and invasion. The present invention is directed to the development of an RT-PCR-based lineage-specific profile as a method to identify the lineages of placental trophoblast cells routinely and specifically. For purposes of the present invention, the four trophoblast cell lineages comprise: (1) trophoblast stem cells (multipotent); (2) trophoblast giant cells (extravillous in humans, invasive); (3) spongiotrophoblast cells (rodent only); and (4) labyrinthine trophoblast cells (syncytiotrophoblasts in humans). Lineages (1), (2) and (4) have direct relevance to and perform similar functions in the human.

One embodiment of the present invention is directed to a method of comprehensively identifying a specific placental cell lineage of a placental cell sample. The method comprises: (1) isolating RNA of the cell sample to yield a total RNA isolate and subjecting the total RNA isolate to reverse transcription PCR (RT-PCR) to yield a placental cell genetic sample; (2) dividing the genetic sample into at least four portions, including a first, a second, a third and a fourth portion; (3) testing the first portion for presence of a trophoblast stem cell lineage marker; (4) testing the second portion for presence of a trophoblast giant cell lineage marker; (5) testing the third portion for presence of a syncytiotrophoblast/labyrinthine cell lineage marker; (6) testing the fourth portion for DNA contamination; (7) conducting an analysis of the results wherein the specific placental cell lineage is determined. A person of ordinary skill in the art will appreciate that step (1) may be readily achieved using one of several technologies commonly known in the art. It is also understood that reverse transcriptase-PCR is sometimes referred to as real time-PCR, and that both are commonly and interchangeably referred to by the RT-PCR acronym. It is also understood that steps 3-6 may include testing/coamplification of, for example, beta Actin or GADPH, as internal controls. “Isolating” is understood to be the equivalent of providing the genetic material in suitable amounts for investigation, such as via amplification, and does not necessarily require isolation

According to a specific embodiment, the presence of a lineage marker is detected by employing trophoblast cell lineage-specific PCR primers. A suitable list of such primers is set forth in Table 3, although any primer which functions to detect the specified marker is also suitably employable and within the scope of the present invention.

In another specific embodiment, the trophoblast stem cell lineage marker is selected from the group consisting of Id2, Cdx2 and ERRbeta. In yet another specific embodiment, the invasive extravillous/trophoblast giant cell marker is selected from the group consisting of Hand1, Mash2, P1-1 and Stra13, and according to a further specific embodiment, the syncytiotrophoblast/labyrinthine cell lineage marker is selected from the group consisting of Tfeb, Tec, Gcm-1, D1x-3, and Esx-1. Although specific genetic markers are expressly provided, the invention is contemplated to include all markers which meet the functional requirement that they designate genes which are uniquely expressed by a particular trophoblast cell lineage, and the inventive method comprises any and all permutations according to the selection scheme. In a very specific embodiment with particular relevance to human placental cell samples, the trophoblast stem cell lineage marker comprises Id2, the trophoblast giant cell marker comprises Stra13, and the syncytiotrophoblast/labyrinthine cell lineage marker comprises Gcm-1.

PCR primers which may be suitably employed in the present invention are set forth in Table 3. Table 3 lists the genetic marker along with one set of PCR primers capable of detecting that marker. It is understood that other primers may be manufactured that enable detection of a particular genetic marker, and that such manufacture is routine to one of ordinary skill in the art. Placental cell samples may derive from a variety of species, including human, and the markers should be selected to enable comprehensive determination of cell lineage for a given species. Positive identification combined with positive elimination of alternatives, in the same test, provides a cost-effective and time-efficient method for determining placental cell lineage of a sample.

Another embodiment of the present invention is directed to a panel of PCR primer pairs suitable for detecting at least one placental cell genetic lineage marker from each of the following: trophoblast stem cell lineage markers, trophoblast giant cell markers, and syncytiotrophoblast/labyrinthine cell lineage markers. According to a specific embodiment of the present inventive panel, the trophoblast stem cell lineage marker is selected from the group consisting of Id2, Cdx2 and ERRbeta, the trophoblast giant cell marker is selected from the group consisting of Hand1, Mash2, Pl-1 and Stra13, the syncytiotrophoblast/labyrinthine cell lineage marker is selected from the group consisting of Tfeb, Tec, Gcm-1, D1x-3, and Esx-1. In a more specific embodiment, the trophoblast stem cell lineage marker comprises Id2, the trophoblast giant cell marker comprises Stra13, and the syncytiotrophoblast/labyrinthine cell lineage marker comprises Gcm-1. The panel may also include at least one PCR primer pair suitable for detecting a spongiotrophoblast cell lineage marker. This panel is particularly relevant for non-human species. Any PCR primer pair suitable for detecting a unique marker may be employed. Table 3 sets forth suitable exemplary primer pairs for each trophoblast cell form.

A further embodiment is directed to a kit which may be used to monitor pregnancy over the course of a pregnancy, or, for example, to detect placental abnormalities early in pregnancy in order to provide opportunity for effective early intervention. Such a kit may be employed in the animal husbandry arts to minimize pregnancy loss, increase successful parturition and to increase overall newborn health. Atypical trophoblast differentiation is known to correlate to several placental failures, including, for example, intrauterine growth retardation and/or preeclampsia. In another embodiment, the methods and kits may be employed to monitor placental cell differentiation as an technique for early detection of placental/trophoblast cancers.

Another embodiment is directed to a method for comprehensively identifying a specific placental cell lineage of a placental cell sample. In this embodiment, it is understood that comprehensive identification may be possible by testing for the presence of as few as two unique markers, depending on, for example, the species, the cell form, the primer design, and the marker being detected. The method comprises: (1) isolating RNA of the cell sample to yield a total RNA isolate and subjecting the total RNA isolate to reverse transcription PCR (RT-PCR) to yield a placental cell genetic sample; (2) dividing the genetic sample into a plurality of portions; (3) testing a portion for presence of a trophoblast stem cell lineage marker; (4) testing a portion for presence of at least one of a trophoblast giant cell lineage marker, and/or a syncytiotrophoblast/labyrinthine cell lineage marker, and/or a spongiotrophoblast cell lineage marker; (5) testing a portion for presence of DNA contamination; and (8) conducting an analysis of the results wherein the specific placental cell lineage is determined.

### EXAMPLES

The following example illustrates analysis and identification of placental trophoblast cell lineages according to one embodiment of the present invention.

The inventive profiling method was used to analyze the mouse SM10 and rat HRP-1 cell lines, isolated from a region of the placental labyrinth, but of previously unidentified lineage. Using this profile, the expression of trophoblast stem cell markers was detected in the SM10 and HRP-1 cells. In contrast, no expression of a marker of differentiated labyrinthine trophoblast was detected. Additionally, both cell lines expressed labyrinthine trophoblast-specific genes and did not express lineage-specific markers of spongiotrophoblasts or trophoblast giant cells. This suggests that SM10 and HRP-1 cell lines are trophoblast stem cell-like cell lines that can be maintained in undifferentiated but committed state in cell culture. These cell lines express labyrinthine-specific genes and are committed to differentiate solely into functional labyrinthine trophoblasts.

The inventive profiling method provides a new technique to identify stem cells and their lineage-specific differentiation. The novel method was further employed to additionally indicate that SM10 and HRP-1 cell lines provide new systems for future studies of stem cell differentiation, allowing investigation of basic mechanisms of differentiation, which may provide insights into the biophysics of development of a specialized system. This method is also useful for identification of other stem cell lines and examination of lineage-specific commitment.

Mouse trophoblast stem cell lines are capable of differentiating into all placental lineages simultaneously, while the rat Rcho-1 trophoblast cell line propagates as stem cells, differentiates into the trophoblast giant cell lineage, and is analogous to invasive human extravillous trophoblasts. The present inventors recently determined that the mouse SM-10 cell line can be propagated as a stem cell population, differentiates into the placental labyrinthine lineage, and is analogous to human syncytiotrophoblasts. The mechanisms, genes, and functions that can be studied in Rcho-1 and SM-10 cells are present and functionally conserved in the human placenta cell lineages. Therefore, the molecular mechanisms regulating trophoblast stem cell growth and differentiation in these cells provide model systems that have direct relevance and applicability to the events that occur in the human placenta. Identifying the signal transduction pathways that regulate trophoblast cell development provides enhanced understanding of the molecular mechanisms that control stem cell growth and differentiation. This new knowledge will provide new insights into potential causes, diagnosis, and treatments of preeclampsia and intrauterine growth retardation.

To evaluate the trophoblast lineage of the SM10 cells, an array of unique trophoblast lineage-specific PCR primers were designed (Table 1). Each of the genes listed in Table 1 has been shown to be exclusively expressed in specific trophoblast lineages during placental formation.

TABLE 1
Lineage-Specific Markers of Trophoblast Gene Expression

| | | | PCR Primer |
|---|---|---|---|
| Gene | Species | Expression | Reference |
| Id2 | Rodent/Human | Trophoblast Stem Cells (M) & | 97 |
| | | Villous Cytotrophoblasts (H) | |
| Dlx3 | Rodent/Human | Labyrinthine (M) & | T L Brown |
| | | Syncytiotrophoblasts (H) | (unpublished) |
| Esx1 | Rodent/Human | Labyrinthine cells | 98 |
| Tfeb | Rodent | Labyrinthine cells | 99 |
| Tec | Rodent | Labyrinthine cells | 100 |
| Gcm1 | Rodent/Human | Syncytiotrophoblasts | 101 |
| 4311 | Rodent | Spongiotrophoblasts | 27 |
| PL1 | Rodent | Trophoblast Giant Cells | 102 |
| Stra13 | Rodent/Human | Trophoblast Giant Cells (M) & | T L Brown |
| | | Extravillous Trophoblasts (H) | (unpublished) |
| Hand1 | Rodent | Trophoblast Giant Cells (M) & | 103 |
| | | Extravillous Trophoblasts (H) | |

The genes set forth in bold represent genes that are present and restricted to specific lineages in both the mouse and human placenta (see Table 1). Analysis of gene expression in proliferative mouse SM10 cells express genes only present in the labyrinthine trophoblast lineage (D1x3, Esx-1, Tfeb, and Tec,). In addition, the D1x3 protein is present in proliferative SM10 cells as determined by Western blotting. Data indicates that the SM10 cell line can be maintained and passaged in culture in a proliferative state, and expresses a major stem cell regulator and lineage markers of labyrinthine trophoblast cells.

Table 2 sets forth exemplary primers according to the present invention.

TABLE 2
PCR PRIMER INFORMATION

| | Ext. | Expect. | Expressed | | | |
|---|---|---|---|---|---|---|
| Primer | tmp | Length | in | control+ | control− | Notes |
| Cdx2 | | 542 bp | Ts3.5 | | | |
| Mash2 | | 369 bp | Ts3.5 | | | |
| Id2 | 62 C. | 497 bp | Sm10 | TS3.5 | | |
| Actin | Any | 243 bp | All | — | — | |
| Tfeb | 62 C. | 297 bp | Sm10 | | | |
| Tec | 62 C. | 663 bp | Sm10 | | | |
| Gcm-1 | 64 C. | ˜700 bp | Sm10 +B | Brain RNA | | New primers |
| Dlx-3 | 64 C. | 408 bp | Sm10 | | Hrp-1 | |
| Glut1 | 62 C. | 697 bp | Sm10 | Hrp-1 | | |
| Glut3 | 56 C. | 527 bp | Sm10 | Hrp-1 | | |
| Glut4 | ˜60 C. | 870 bp | Sm10 | | | |
| 4311 | 58 C. | 458 bp | Sm10 | TS3.5 | Sm10 | |
| Esx-1 | 56 C. | 254 bp | Sm10 | TS3.5 | Rcho-1 | No actin simult. |
| Mekk3 | | 562 bp | | | | |
| mGCNF | | 239 bp | | | | |
| PL-1 | 62 C. | 746 bp | Rcho-1 | Rcho-1 | Sm10 | |
| HIF1-alpha | 58 C. | 373 bp | Rcho-1 | | | |
| Hand-1 | 64 C. | 746 bp | Rcho-/always | Ts3.5 | Sm10 | |
| mmp-9 | 55 C. | 769 bp | Rcho-1/D7diff | | | |
| Stra 13 | 62 C. | 596 bp | Rcho-1/D7diff | | | |
| mSna | 62 C. | 496 bp | Rcho-1/D3diff | | | |

The ability to specifically establish cell lineage was confounded by the lack of a cell line that would represent a suitable model of trophoblast stem cell-like cells capable of differentiating into the “transport” phenotype characteristic of human syncytiotrophoblasts or murine labyrinthine trophoblasts.

Hence, the present inventors investigated and recently identified a mouse cell line that serves as such a model, the mouse SM10 cell line. The SM10 cell line was provided by Dr. Joan Hunt, Kansas University Medical Center. SM10 cells were isolated from the labyrinthine region of the mouse placenta and established from explant outgrowths. Although of trophoblast origin, the specific identity of these cells, with regards to trophoblast lineage, has not been reported. Our preliminary data indicate that SMIO cells can be propagated as stem cell-like cells for many passages in cell culture. RT-PCR analysis using lineage and gene specific primers indicated that proliferative SM10 cells express a major inhibitor of cellular differentiation, the Inhibitor of differentiation 2 (Id2) gene. Id2 is a specific lineage marker of trophoblast term cells in humans and mice. As shown in FIG. 2, SM10 cells express Id2. As expected, Id2 expression was also confirmed in proliferative, mouse trophoblast stem (TS_{3.5}) cells provided by Dr. Janet Rossant, Mount Sinai Hospital. Our results indicate that SM10 cells express Id2 and represent a trophoblast “stem cell-like” cell line.

To further evaluate the trophoblast lineage of the SM10 cells, we designed an array of unique trophoblast lineage-specific PCR primers (Tables 2 and 3). Each of the genes listed in Table 2 has been shown to be exclusively expressed in specific trophoblast lineages during placental formation.

The genes (Table 1), in bold, represent genes that are present and restricted to specific lineages in both the mouse and human placenta. Analysis of gene expression in proliferative SM10 cells by RT-PCR indicates, that in addition to Id2, proliferative mouse SM10 cells express genes only present in the labyrinthine trophoblast lineage (D1x3, Esx1, Tfeb and Tec). In addition, the D1x3 protein is present in proliferative SM10 cells as determined by Western blotting. The data indicates that the SM10 cell line can be maintained and passaged in culture in a proliferative state, and expresses a major stem cell regulator and lineage markers of labyrinthine trophoblast cells.

Further lineage analysis on proliferative SM10 cells by RT-PCR using specific markers for spongiotrophoblast and “invasive” giant cell lineages, including spongiotrophoblast (4311) and giant cells (Hand1), were performed. Differentiated Rcho-1 cells express markers that are specifically present in trophoblast giant cells (PL1, Stra13). The data demonstrates that SM10 cells do not express genes of the spongiotrophoblast or trophoblast (invasive) giant cell lineages.

The lineage analysis indicates that the SM10 cell line is representative of proliferative trophoblast stem cells that will be committed to the labyrinthine lineage if differentiated. To further analyze SM10 and other placental trophoblast cell lines, several cell lines were treated with TGFβ, a major regulator of placental development, to determine the impact on trophoblast cell growth.

Trophoblast stem cell growth in serum containing media was inhibited in SM10 cells (40%) and TS cells (20%) in the presence of TGFβ. In SM 10 cells, dose response analysis indicated that TGFβ was maximally growth inhibitory at doses equal to or greater than 1 ng/ml (˜40 pM) (data not shown), consistent with physiological levels of TGFβ found in vivo. In addition, TGFβ specifically induced growth inhibition, as determined by TGFβ antibody neutralization experiments analyzing cell growth. The data demonstrates that TGFβ specifically inhibits the proliferation of SM10 cells.

TGFβ mediated growth inhibition in SM10 cells suggested that the cells may be growth arrested or undergoing apoptosis (cell death). SM10 cell viability, determined by trypan blue exclusion, indicated that in the present of TGFβ, nearly 100% of SMIO cells remained viable up to 10 days.

To further analyze the effects of TGFβ, SM10 cells were treated and examined morphologically. The data indicates that TGFβ induces a complete and dramatic morphological aggregation of nonproliferative SM10 cells with cellular fusion within 72 h and this can be maintained for at least 10 days in the presence of TGFβ. The results indicate that TGFβ induces the differentiation of trophoblast stem cell-like cells at the cellular (morphological) level.

Although TGFβ was capable of inducing substantial morphological aggregation and apparent cellular fusion, we examined a more definitive marker of fusion in labyrinthine or syncytiotrophoblasts, Gem-1. Gem-1 is the definitive lineage marker for and is expressed in the placenta only in differentiated human syncytiotrophoblasts and mouse labyrinthine trophoblasts. SM10 cells treated with TGFβ for 72 h dramatically induced the expression of Gem-1, coincident with morphological differentiation.

The Id proteins are major regulators of stem cells and cellular differentiation. Because SM10 cells express Id2 in the proliferative, stem cell-like state, it was determined whether TGFβ-induced differentiation could inhibit Id2 expression. In conjunction with the induction of Gem-1 expression, a corresponding loss of Id2 mRNA in the presence of TGFβ in SM10 cells was detected. The results of trophoblast lineage analysis at the molecular level indicate that TGFβ induces the differentiation of the SM10 trophoblast stem cell-like cells specifically to labyrinthine/syncytial trophoblasts.

TABLE 3

Exemplary PCR Primer Pairs suitable for detecting Placental cell
lineage genetic markers

| | | |
|---|---|---|
| Gene | Forward | Reverse |

Trophoblast stem cell lineage markers

| | | |
|---|---|---|
| Id2 | 5′tctgagcttatgtcgaatgatagc3′ | 5′cacagcattcagtaggctcgtgtc3′ |
| Cdx2 | 5′cccagcggccagcggcgaaacctg3′ | 5′ttctcgcagcgtccatactcctcat3′ |
| ERRBeta | 5′tcaagtgcgagtacatgctt3′ | 5′gaaatctgtaagctcaggta3′ |

Invasive extravillous (human) and trophoblast giant cells (rodent)

| | | |
|---|---|---|
| Hand1 | 5′gcgcctggctaccagttaca3′ | 5′agcaacgccttccctctagg3′ |
| Mash2 | 5′gaaggtgcaaacgtccacttc3′ | 5′ccttactcagcttcttgttgg3′ |
| PL-1 | 5′tgactttgactctttcgggct3′ | gctctgaatacaccgagagcg3′ |
| Stra13 | 5′tttccagachtgtgccc3′ | 5′taccagcayttctccagca3′ |

Spongiotrophoblast lineage (rodent)

| | | |
|---|---|---|
| 4311/TPBP | 5′caggtacttgagacatgactc3′ | 5′ggcagagatttcttagacaatg3′ |

Syncytiotrophoblasts (human) and labyrinthine (rodent)

| | | |
|---|---|---|
| Tfeb | 5′gcgcatgcagcagcaggctgtc3′ | 5′ctggggatgctgctggggcagg3′ |
| Tec | 5′ataagaaagaccctgcctccc3′ | 5′aagcctcaccactccaaaca3′ |
| Gcm-1 | 5′agaccaagctggaagcagag3′ | 5′gcatgttgctgtgagtagg3′ |
| Dix-3 | 5′atctcaatgggctcgcag3′ | 5′atggagtcactgttgttggg3′ |
| Esx-1 | 5′gcaaccccaacaggagc3′ | 5′ggactcatggcgactgga3′ |

Internal Controls

| | | |
|---|---|---|
| Beta Actin | 5′atcgtgggccgccctaggca3′ | 5;tggccttagggttcagaggg3′ |
| GAPDH | 5′ggagtcaacggatttggt3′ | 5′gtgatgggatttccattgat3′ |

What is claimed:

1. A method of comprehensively identifying a specific placental cell lineage of a placental cell sample, the method comprising:
(1) isolating RNA of the cell sample to yield a total RNA isolate and subjecting the total RNA isolate to reverse transcription PCR (RT-PCR) to yield a placental cell genetic sample;
(2) dividing the genetic sample into at least four portions, including a first, a second, a third and a fourth portion;
(3) testing the first portion for presence of a trophoblast stem cell lineage marker;
(4) testing the second portion for presence of a trophoblast giant cell lineage marker;
(5) testing the third portion for presence of a syncytiotrophoblast/labyrinthine cell lineage marker;
(6) testing the fourth portion for DNA contamination;
(7) conducting an analysis of the results wherein the specific placental cell lineage is determined.

2. The method according to claim 1, wherein the presence of a lineage marker is detected by employing trophoblast cell lineage-specific PCR primers.

3. The method according to claim 1, wherein the trophoblast stem cell lineage marker is selected from the group consisting of Id2, Cdx2 and ERRbeta.

4. The method according to claim 1, wherein the trophoblast giant cell marker is selected from the group consisting of Hand1, Mash2, Pl-1 and Stra13.

5. The method according to claim 1, wherein the syncytiotrophoblast/labyrinthine cell lineage marker is selected from the group consisting of Tfeb, Tec, Gcm-1, D1x-3, and Esx-1.

6. The method according to claim 1 wherein steps 3-6 include testing for an internal control.

7. The method according to claim 6 wherein the internal control comprises Beta Actin or GADPH.

8. The method according to claim 1 wherein the trophoblast stem cell lineage marker comprises Id2, the trophoblast giant cell marker comprises Stra13, and the syncytiotrophoblast/labyrinthine cell lineage marker comprises Gcm-1.

9. The method according to claim 2, wherein the trophoblast cell lineage-specific PCR primers are selected from Table 3.

10. The method according to claim 1, wherein the placental cell sample derives from a human.

11. A panel of PCR primer pairs suitable for detecting at least one placental cell genetic lineage marker from each of the following: trophoblast stem cell lineage markers, trophoblast giant cell markers, and syncytiotrophoblast/labyrinthine cell lineage markers.

12. The panel according to claim 11 wherein the trophoblast stem cell lineage marker is selected from the group consisting of Id2, Cdx2 and ERRbeta, the trophoblast giant cell marker is selected from the group consisting of Hand1, Mash2, Pl-1 and Stra13, the syncytiotrophoblast/labyrinthine cell lineage marker is selected from the group consisting of Tfeb, Tec, Gcm-1, D1x-3, and Esx-1.

13. The panel according to claim 12, wherein the trophoblast stem cell lineage marker comprises Id2, the trophoblast giant cell marker comprises Stra13, and the syncytiotrophoblast/labyrinthine cell lineage marker comprises Gcm-1.

14. The panel according to claim 11, wherein the panel further comprises at least one PCR primer pair suitable for detecting a spongiotrophoblast cell lineage marker.

15. The panel according to claim 11, wherein the trophoblast cell lineage-specific PCR primers are selected from Table 3.

16. A kit for monitoring pregnancy, the kit comprising the panel of claim 14.

17. The kit according to claim 16 further comprising PCR primer pairs suitable for detecting beta-actin and GAPDH as internal controls.

18. A method for monitoring pregnancy, the method comprising employing the method according to claim 1 to detect atypical trophoblast differentiation.

19. The method according to claim 18, wherein the atypical trophoblast differentiation is associated with intrauterine growth retardation and/or preeclampsia.

20. A method of comprehensively identifying a specific placental cell lineage of a placental cell sample, the method comprising:
(1) isolating RNA of the cell sample to yield a total RNA isolate and subjecting the total RNA isolate to reverse transcription PCR (RT-PCR) to yield a placental cell genetic sample;
(2) dividing the genetic sample into a plurality of portions;
(3) testing a portion for presence of a trophoblast stem cell lineage marker;
(4) testing a portion for presence of at least one of a trophoblast giant cell lineage marker, a syncytiotrophoblast/labyrinthine cell lineage marker, a spongiotrophoblast cell lineage marker;
(7) testing a portion for presence of DNA contamination;
(8) conducting an analysis of the results wherein the specific placental cell lineage is determined.
